(19) United States
(12) Patent Application Publication
(10) Pub. No.: US 2007/0007159 A1
(43) Pub. Date: Jan. 11, 2007

(54) Container for surgical instruments

(75) Inventor: Jean-Albert Despres, Souesmes (FR)

Correspondence Address: LEYDIG VOIT & MAYER, LTD, 700 THIRTEENTH ST. NW, SUITE 300, WASHINGTON, DC, 20005-3960, US

(73) Assignee: CYRIL DE UTHEMANN, Vandoeuvres (CH)

(21) Appl. No.: 11/478,374

(22) Filed: Jun. 30, 2006

(30) Foreign Application Priority Data
Jul. 4, 2005 (FR) 05 07 080

(51) Int. Cl. A61B 19/02; B65D 1/34; A61B 17/06

(52) U.S. Cl. 206/439; 206/438; 206/557; 206/565

(57) ABSTRACT

A container, particularly for surgical instruments in the form of tray, which has a rigid frame, the bottom of which is made of a replaceable material, advantageously a single-use material, such as paper or fabric.

The invention relates to a container, particularly for surgical instruments

The objective of the invention is to propose instruments of this type that is easy to manipulate and that satisfies the most stringent requirements of hygiene.

To attain this objective, a container according to the invention is produced in the form of a tray with a rigid frame, the bottom of the tray being made of a replaceable material, advantageously a single-use material such as paper or fabric.

According to one characteristic of the invention, the rigid frame is formed by superposition of two basic frames, enclosing between them the edges of the bottom element so that it forms a bin for housing an instrument inside the frame.

According to another characteristic of the invention, the basic frames and the bottom element are rectangular in shape, and the bottom element is inserted between the two frames along two opposite sides.

According to yet another characteristic of the invention, the tray has an arch at the free end of each bin, which arch is attached to the lower frame to support the curved edge of the bottom element in the form of a bin.

According to yet another characteristic of the invention, the space between the lower frame element and the support arch is closed by a wall for laterally closing off the bin.

According to yet another characteristic of the invention, the tray has a number of juxtaposed bins.

According to yet another characteristic of the invention, the frames are produced out of a rigid material that is easy to clean, such as stainless steel.

Thanks to this configuration of the containers according to the invention, they not only are easy to manipulate and conform to the most stringent requirements of hygiene, but also they can be used in the context of an entirely automatic process or installation for sorting instruments, particularly surgical instruments.

The invention will be better understood, and other objectives, characteristics, details and advantages of it will become more apparent in the course of the following explanatory description in reference to the appended diagrammatic drawings, given only by way of example, illustrating an embodiment of the invention, and in which:

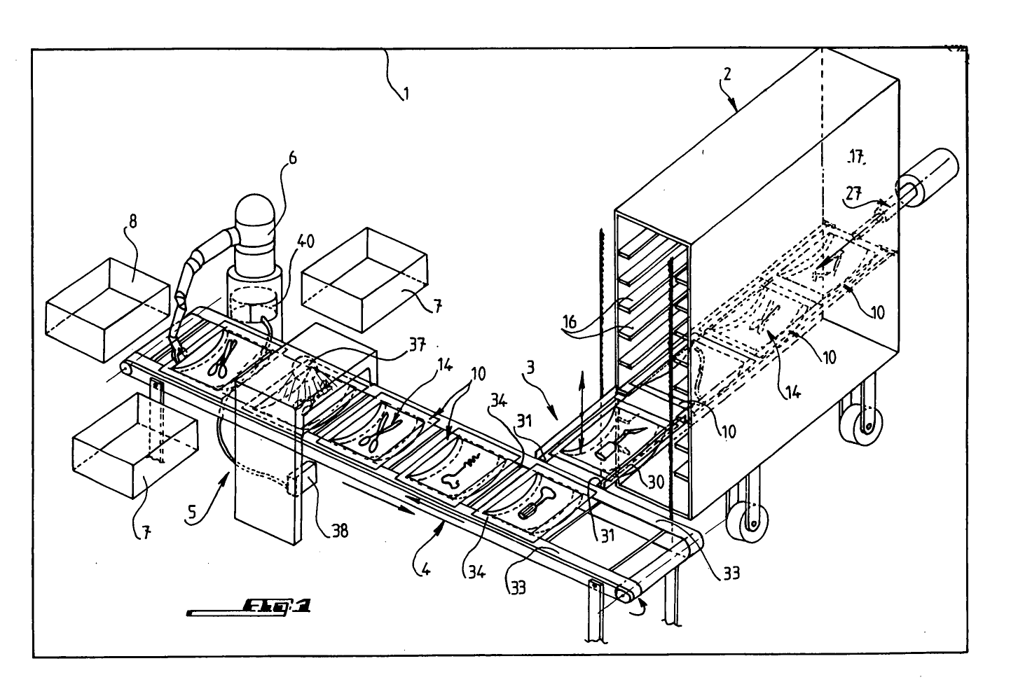

FIG. 1 is an oblique schematic view of an installation according to the invention;

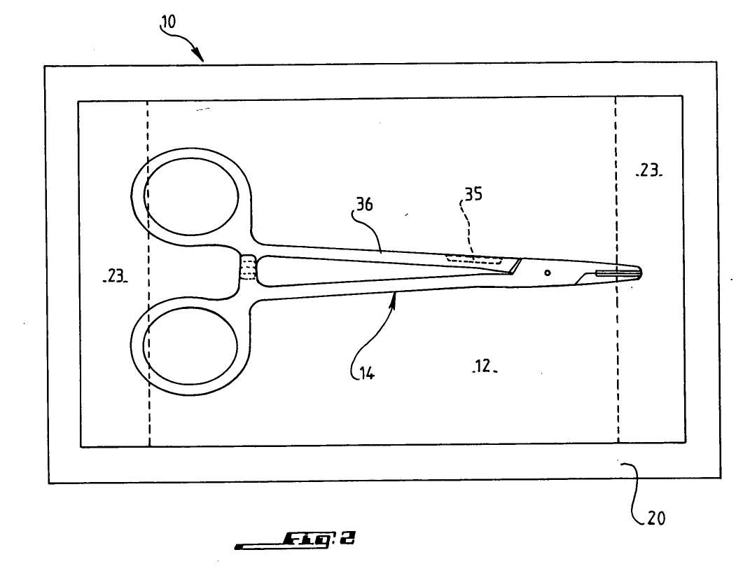

FIG. 2 is a top view of an instrument support tray according to the invention;

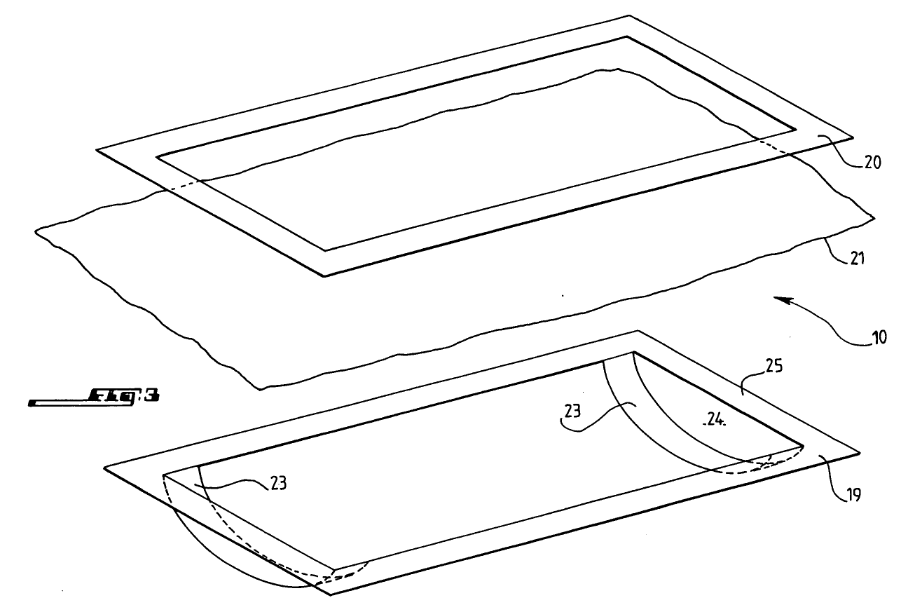

FIG. 3 is an exploded view of a support tray according to FIG. 2; and

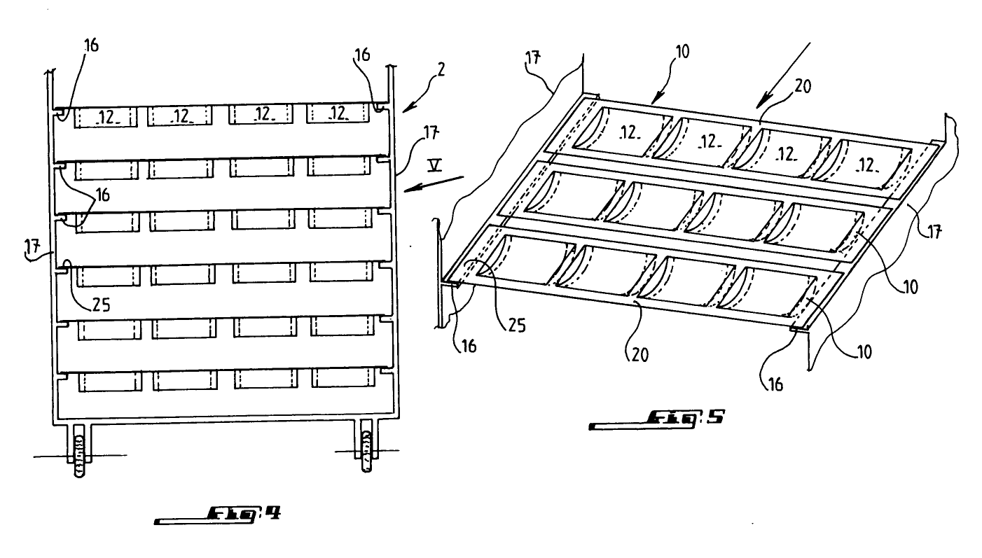

FIGS. 4 and 5 illustrate an advantageous configuration of the trays and of storage shelving according to the invention.

So that the advantages and particularities of the containers according to the invention can be appreciated, they are described hereafter in the context of their use in an automatic surgical instrument sorting process and installation. The description is of course given only by way of example, but the invention can generally, be used for any operation involving a specific selection and for stowing instruments for implementation of the operation.

In reference to FIG. 1, it is observed that an installation according to the invention for sorting the instruments necessary for a surgical operation essentially entails, arranged inside of enclosure 1 with a pure atmosphere, that is, of the most perfect cleanliness possible, essentially a device for storage of all the surgical instruments likely to be used for the various possible surgical operations, in the clean state, device 3 for transfer of the instruments to conveyor 4 intended for transporting them to station 5 for identification of the nature or type of instruments, and a mechanism for stowing the instruments after their identification in containers 7, each of which is intended to hold the instruments to be used for a pre-determined operation, container 8 being provided for receiving instruments considered not to correspond to the requirements established for the surgical operations.

Device 2 for storage of all of the instruments is executed in the example in the form of a carriage with a certain number of levels 9, each for holding a number of trays 10, three trays in the example represented, each of which can have a number of bins 12 for housing surgical instrument 14. In this specific case, to simplify the drawings, each tray only has one bin. Trays 10 of each level 9 of storage carriage 2 are supported by support elements in the form of slide rails 16, each attached to lateral wall 17 of the carriage oriented in the direction of conveyor 4.

In reference to FIG. 3, it is observed that each tray 10 is composed of two superposed frames made of a material that is easy to clean, such as stainless steel, namely lower frame element 19 with a general rectangular shape and upper frame 20 of a complementary shape that can be attached on the lower frame by means of clips (not represented) that can squeeze the upper frame onto the lower frame so that replaceable element 21, advantageously a single-use element made of flexible material transparent to X-rays, such as paper or fabric, could be inserted at two of its opposite edges between the corresponding edges of the two frames in order to form bin 12 for housing instrument 14, and thus the bottom of the tray. For easy formation and maintaining of the bins, lower frame 19 is provided with support elements 23 in the form of arches at each longitudinal end. It is advantageous for the bin to be closed at each longitudinal end by vertical wall 24 forming the space between the frame and the corresponding arch 23.

As seen in FIG. 1, each tray 10 can be moved in carriage 2 perpendicular to its longitudinal axis, resting by its short sides 25 on slide rails 16 of the carriage.

In order to push the trays out of the carriage, the arrangement for transfer of trays 10 of each stage of the carriage incorporates pushing device 27 mounted to be vertically mobile at the rear of carriage 2, so that it can be positioned at each level 9 of the carriage. The device has a pushing piston of a hydraulic cylinder which, during its outward movement, pushes the last tray and thus moves all of the trays toward the conveyor.

Moreover, transfer device 3 has, in front of storage carriage 2 for trays 10, frame 30 for transfer of trays 10 from carriage 2 to conveyor 4, which frame can be moved vertically so that it can receive the trays of each storage level 9 of the carriage in order to transport them to the height of conveyor 4 so that the trays can be put on the conveyor. More precisely, in the example represented, the transfer frame essentially has two slide rails 31, each capable of being aligned, in a position for receiving a tray from level or stage 9, with slide rail 16 of that level, so that trays 10 can be moved under the effect of pushing device 27 from rails 16 of the carriage to rails 31 of transfer frame 30. The transfer frame is dimensioned to successively transfer trays 10 to conveyor 4.

Conveyor 4 is shown in FIG. 1 as produced in the form of an endless conveyor belt with essentially two parallel flat belts 33, connected by crosspieces 34, for the transport of trays 10.

In order to ensure the transfer of trays 10 from transfer frame 30 to conveyor belts 33, support rails 31 of the transfer frame extend to the point at which they are over the conveyor, and its tray support portion, in the position before transfer, can be lowered in order to allow the carriages to be placed over the belts of the conveyor situated below. Then they move away laterally and return to their position for receiving another tray.

Conveyor 4 transports the trays received from storage carriage 2 via transfer frame 3 to station 5 for identification of the instruments placed in bins 12 of trays 10.

Concerning the surgical instruments, which have different functions and shapes, each of them is provided with an identification code which can be recognized by station 5 so that the station can distinguish the instruments brought in by conveyor 4 according to their specific type. More precisely, the identification code of each instrument is marked in any appropriate form on insert 35 which is placed in an appropriate cavity machined in the instrument and then closed. FIG. 2 shows, as an example, surgical pliers with insert 35 incorporated in one of its branches labeled 36.

The station for identification of the instruments is advantageously an X-ray reader in the form of a portal through which conveyor 4 passes and which has, arranged above conveyor 4, X-ray source 37, while a receiver of the rays passing through instrument 14 is arranged below the conveyor.

So that it is possible to recognize the type of an instrument according to its insert, the elements of the insert that form the identification code must be less transparent to X-rays than the material constituting the instrument. This code could reside, for example, in the shape of the insert or shape of the marks provided on it or else the shape or placement of a notch made in the insert. Since inserts of this type are known, it is not necessary to describe it in more detail. The inserts are advantageously produced out of a material which is relatively opaque to X-rays. They could be made out of brass or an alloy based on brass, while the instruments are made out of stainless steel. Generally, for the choice of the materials, the heavier the atoms constituting the material, that is, the higher their atomic weight, the more they absorb X-rays and therefore the more opaque the material is to X-rays. In order to ensure reliable identification of the instruments, it is necessary for the instruments always to be presented at the identification station in a clearly defined position.

After identification of the instruments by reading of their inserts using X-rays, stowing apparatus 6 grasps the instruments and stows them in containers 7, in the form of boxes, according to the orders of information processing device 40.

This device has operating protocols in its memory, one protocol for each type of operation, indicating which instruments are to be used in the course of an operation, if applicable, in their order of use. Given that each type of operation corresponds to a box 7, the instruments to be stowed in this box are indicated by the protocol established for this operation. For this purpose, information processing device 40 first identifies, according to the signal that it has just received from reader 38 of the identification station, the type of instrument that was just examined, and then determines, by referring to the various protocols, which operation and thus which box 7 an instrument of this type is intended for. It then gives the order to stowing device 6 to grasp the identified instrument in the tray and to stow it in the appropriate box.

By comparing the instruments stowed in a box with those appearing in the protocol, the information processing device knows at any time the “filling” state of each box 7. If it establishes that a box is complete, that is, it contains all the instruments necessary for the operation at issue, the box is closed, for example, by putting its cover on.

The invention also provides for the possibility of separating instruments considered unfit for use from the usage circuit, by placing them in discard box 8. There could be various reasons for this measure; for example, because an instrument is worn, an instrument is impossible to identify, or an instrument is soiled.

Concerning stowing station 6, apparatuses capable of functioning in the manner described above are generally known, so that it is not necessary to describe precisely the apparatus used in the context of the invention. It is appropriate to indicate that such an apparatus has a robot arm capable of grasping the instruments in their tray and then placing them in the appropriate boxes as a function of the instructions received from the information processing device.

Concerning the functioning of the invention and the running of the process as well as the different steps of the process, they proceed from the description just given. It goes without specifying further that for each transfer of a tray onto the conveyor, the conveyor is stopped briefly, for the time necessary to place the tray. The stops of the conveyor for loading the trays and the process of identification of the instruments by the identification station, as well as the stowing of the instruments, are coordinated by the information processing device.

The description of the invention just given has been given only as an example, and multiple modifications can be made without departing from the scope of the invention. In order to increase the storage capacity of carriage 2, each tray 10 could have four bins as shown by FIGS. 4 and 5. The arrow symbolizes the action of the mechanism for moving the tray towards conveyor 3.

1. A container, for holding surgical instruments, and including a tray that has a rigid frame with a bottom of a replaceable material selected from the group consisting of paper and fabric.

2. A The container according to claim 1, wherein the rigid frame includes two superposed basic frames enclosing between them edges of a bottom element, forming a bin for housing a surgical instrument inside the rigid frame.

3. The container according to claim 2, wherein the basic frames and bottom element are rectangular in shape, and the bottom element is inserted between the two basic frames along two opposite sides.

4. The container according to claim 2, wherein the tray has, at free ends of the bin, an arch, which is attached to a lower one of the basic frames, supporting a curved edge of the bottom element.

5. The container according to claim 4, wherein space between the lower one of the basic frames and the support arch is closed by a wall laterally closing the bin

6. The container according to claim 2 wherein the tray includes a plurality of juxtaposed bins.

7. The container according to claim 2, wherein the two basic frames are stainless steel.

8. The container according to claim 3, wherein the tray has, at free ends of the bin, an arch which is attached to a lower one of the basic frames, supporting a curved edge of the bottom element.

9. The container according to claim 8, wherein a space between the lower one of the basic frames and the support arch is closed by a wall laterally closing the bin.
